(19) United States
(12) Patent Application Publication
(10) Pub. No.: US 2022/0105025 A1
(43) Pub. Date: Apr. 7, 2022

(54) Hair and Scalp Treatment Solution

(71) Applicant: Ann Isidore, New Orleans, LA (US)

(72) Inventor: Ann Isidore, New Orleans, LA (US)

(21) Appl. No.: 17/143,215

(22) Filed: Jan. 7, 2021

Related U.S. Application Data
(60) Provisional application No. 63/088,487, filed on Oct. 7, 2020.

(51) Int. Cl. A61K 8/9794; A61K 8/04; A61K 8/31; A61K 8/92; A61K 8/67; A61K 8/36; A61K 8/19; A61K 8/27; A61Q 5/12; A61Q 5/00; A61Q 5/02; A61Q 7/00

(52) U.S. Cl. CPC A61K 8/9794; A61Q 5/006; A61K 8/31; A61K 8/922; A61K 8/673; A61K 8/676; A61K 8/67; A61K 8/361; A61K 8/19; A61K 8/27; A61Q 5/12; A61Q 5/002; A61Q 5/02; A61Q 7/00; A61Q 5/008; A61K 8/042

(57) ABSTRACT

This present invention relates to a hair and scalp treatment and strengthening composition. The hair and scalp treatment and strengthening composition comprises quantities of an aloe vera gel, turmeric root, ginger root, petroleum jelly, grape seed oil, coconut oil, peppermint oil, rosemary oil, a grave sea oil, and vanilla fragrance oil. The hair and scalp treatment and strengthening composition assists in reducing alopecia, treating over processed hair and hair loss, as well as stimulating new hair growth.

### CROSS-REFERENCE TO RELATED APPLICATION

The present application claims priority to, and the benefit of, U.S. Provisional Application No. 63/088,487, which was filed on Oct. 7, 2020 and is incorporated herein by reference in its entirety.

### FIELD OF THE INVENTION

The present invention relates generally to the field of hair care products and treatments. More specifically, the present invention relates to a unique hair and scalp treatment formulation to condition the scalp, stimulate hair follicles, and thicken the hair of both men and women alike. The innovative hair treatment solution assists in reducing alopecia, over processed hair conditions, hair loss and other hair and scalp related issues. In a preferred embodiment, the hair treatment formulation is comprised of various natural ingredients including, without limitation, aloe vera gel, turmeric root, ginger root, grape seed oil, coconut oil, grave sea oil, and more. Accordingly, the present disclosure makes specific reference thereto. Nonetheless, it is to be appreciated that aspects of the present invention are also equally applicable to other like applications, devices and methods of manufacture.

### BACKGROUND OF THE INVENTION

By way of background, there are approximately 100,000 hair follicles on the human head, and healthy hair grows at about 0.2 to about 0.7 inches per month or on average about ½ inch per month or about 6 inches per year. Nonetheless, certain factors such as age, health condition, diet, and hair type can all impact the amount of hair growth that an individual may experience. Each strand of hair has a life span of about 2 to 8 years and after the transition and resting phase, the hair falls out leading to the loss of between 100-150 strands of hair per day.

Notwithstanding the process of natural hair growth and loss, other factors may contribute to premature hair loss. For example, alopecia areata is a common autoimmune skin disease that results in unpredictable hair loss on the scalp, face and sometimes other areas of the body. This skin disorder occurs when an individual's immune system causes white blood cells to attack the cells in hair follicles and cause them to shrink, thereby slowing down or even stopping hair production and growth. Unfortunately, alopecia occurs in males and females of all ages and races, and approximately 6.8 million people in the United States and 147 million worldwide have or will develop alopecia areata at some point during their lives. When afflicted with alopecia an individual not only loses his or her hair in patches, but the disease also causes emotional distress to the individual, as the disease affects the appearance of the individual and may cause the individual to withdraw from social interactions.

In addition to alopecia areata, other scalp and hair related conditions, such as over processed hair, stress, medicinal side-effects, hormonal changes and other medical conditions, may cause damage to the hair and/or scalp and eventually result in hair loss to the individual. By way of example, over processed hair usually occurs when there is an incorrect usage or over usage of harsh chemicals including, without limitation, bleach, hair relaxers, perming solutions and/or heat styling tools, which may result in the hair becoming frizzy and/or having white or split tips on the ends of the hair shaft. When such conditions occur, the afflicted individual will typically attempt to treat his or her hair to prevent further damage, or reverse the current damage, in an effort to return their hair to its previous luster and condition.

Historically, various hair treatment products and services have been used to treat damaged hair, and/or to prevent hair from becoming damaged in the first place. For example, many individuals opt for frequent haircuts to get rid of split ends, frizzy hair and/or dead hair strands caused by over processed hair. However, it is not always feasible to get frequent haircuts, and the same may be expensive. Also, while cutting one's hair may remove the damaged hair, it does not treat the underlying causes of the damaged hair or those portions of the damaged hair that remain on the individual's head. Other solutions include reduction of styling time and/or use of chemical-based products. However, many styling tools and chemical-based products are not as effective when used for less than the recommended time, and may cause further damage to the individual's hair and/or scalp. Still, other individuals may use a heat protectant spray to shield their hair from damage, frizz and dryness. However, many such heat protectant sprays are also comprised of chemical-based ingredients, which may lead to additional damage of the hair over time.

Additionally, a number of products, such as shampoos, lotions, and the like, are currently available to treat hair loss due to a medical condition such as alopecia or the like. While such products claim to cure alopecia, strengthen and thicken the hair, and condition the scalp, many of such products are not only expensive but are also largely ineffective. Specially, it is difficult to find products that effectively stimulate hair growth, and many such products have undesirable side effects. For example, the most common form of alopecia areata treatment is the use of corticosteroids, which are powerful anti-inflammatory drugs that can suppress the immune system. Other medications that can be prescribed that either promote hair growth or affect the immune system include Minoxidil, Anthralin, SADBE, and DPCP. Although some of these may help with the re-growth of hair, they do not always prevent the formation of new bald patches. Additionally, the medicinal treatment of alopecia areata is expensive, may lead to other side effects and cannot be obtained by everyone.

Therefore, there exists a long felt need in the art for a hair and scalp treatment solution which treats hair loss caused by diseases such as alopecia or other medical conditions. There is also a long felt need in the art for a unique hair treatment solution which can be easily applied to the hair and scalp area to effectively enhance the quality of the individual's hair, as well as the condition of the scalp. Additionally, there is a long felt need in the art for a hair treatment solution that prevents hair from becoming damaged due to over processing, and that stimulates hair growth. Moreover, there is a long felt need in the art for a hair treatment solution that is all natural, and free of harsh chemicals and parabens. Finally, there is a long felt need in the art for a hair and scalp treatment solution that is inexpensive to manufacture, and that is cost-effective and affordable to treat hair loss, damaged hair and related conditions.

The subject matter disclosed and claimed herein, in one embodiment thereof, comprises an amber colored hair and scalp strengthening and conditioning composition that comprises aloe vera gel, turmeric root, ginger root, petroleum jelly, grape seed oil, coconut oil, peppermint oil, rosemary oil, vanilla fragrance oil, grave sea oil, and various combinations thereof. The hair and scalp strengthening and conditioning formulation may be in the form of a lotion, cream, gel, conditioner, aerosol (with a proper propellant), mousse or grease that can be applied by a sponge or other suitable applicator.

In this manner, the novel hair and scalp treatment solution of the present invention accomplishes all of the forgoing objectives, and provides a relatively safe, efficient, convenient and cost-effective way of preventing damage to the hair of an individual, as well as to repair damaged hair and treat other conditions such as alopecia and the like. The hair and scalp treatment solution of the present invention is also user friendly, inasmuch as it is less expensive and more effective than its alternatives, contains all natural ingredients, and can be easily applied to the scalp and hair of the user.

### SUMMARY OF THE INVENTION

The following presents a simplified summary in order to provide a basic understanding of some aspects of the disclosed innovation. This summary is not an extensive overview, and it is not intended to identify key/critical elements or to delineate the scope thereof. Its sole purpose is to present some general concepts in a simplified form as a prelude to the more detailed description that is presented later.

The subject matter disclosed and claimed herein, in one embodiment thereof, comprises a hair and scalp strengthening and conditioning formulation that comprises an aloe vera gel, turmeric root, ginger root, petroleum jelly, grape seed oil, coconut oil, peppermint oil, rosemary oil, vanilla fragrance oil, grave sea oil, and combinations thereof. The hair and scalp strengthening and conditioning formulation may be in the form of a lotion, cream, gel, conditioner, aerosol (with a proper propellant), mousse or grease, which may be applied by a sponge or other suitable applicator.

In a further embodiment of the present invention, a method of strengthening and conditioning hair of both men and women is disclosed. The method comprises the step of applying the novel hair and scalp treatment solution of the present invention to an individual's hair and scalp. In one embodiment, the novel hair and scalp treatment solution is comprised of about 3.0% by weight of an aloe vera gel, about 3.0% by weight of turmeric root, about 4.0% by weight of ginger root, about 10.0% by weight of petroleum jelly, about 3.0% by weight of grape seed oil, about 5.0% by weight of coconut oil, about 5.0% by weight of peppermint oil, about 5.0% by weight of rosemary oil, about 7.0% by weight of vanilla fragrance oil, about 3.0% by weight of grave sea oil and the remainder being water. Notwithstanding, other essential oils that may help with relaxation of the scalp and the individual may be added to the formulation including, without limitation, lavender, rose, pumpkin, jojoba, eucalyptus, frankincense, cinnamon leaf, lime, cedarwood and lemongrass. These essential oils may be added in an amount of approximately 0.5% by weight to about 3% by weight, depending on the essential oil added and its potency. The hair and scalp treatment solution may also further comprise vitamin supplements such as, but not limited to, omega 3 and 6, zinc, vitamin C, vitamin D, iron, vitamin B-5 and biotin, which may be added to catalyze hair regrowth. The carrier oils in the hair and scalp treatment solution, such as coconut, rosemary, and peppermint help to dilute the essential oils. Nonetheless, other carrier oils, such as sunflower, castor and mineral, may also be used. The hair and scalp treatment solution may further comprise natural colors (in the form or a dye or the like) to create a particular aesthetic feature so that the hair and scalp treatment solution appears amber, cream colored, tan or any other color that suits user need and/or preference.

In a further embodiment of the present invention, the hair and scalp treatment solution for stimulating hair growth and reducing alopecia is comprised essentially of 15-40% by weight of a nourishing or carrier oil that may include grape seed oil, coconut oil, peppermint oil, rosemary oil and vanilla fragrance oil or a combination thereof, 20-25% by weight of an aloe vera extract, 3-5% by weight of turmeric root, 4-6% by weight of ginger root, and 10-20% by weight of petroleum jelly.

In yet another embodiment of the present invention, a hair growth serum/oil for application to the scalp and hair to repair damaged hair, treat the scalp and promote hair growth is disclosed. The hair growth serum/oil comprises a mixture of aloe vera gel, turmeric root, ginger root, petroleum jelly, grape seed oil, coconut oil, peppermint oil, rosemary oil, and vanilla fragrance oil. The mixture can be tinted as needed to provide a suitable aesthetic characteristic to the formulation, such as an amber, tan or cream color and is desirably used to stimulate hair growth and reduce alopecia. The aloe vera is available in a form that has been processed from the plant, and grape seed extracts are industrial derivatives from whole grape seeds. Peppermint oil is the essence of peppermint extracted into an oil, and the coconut oil is produced from coconut milk by grating the coconut and mixing it with water, then squeezing out the oil which acts as an emollient. Finally, the rosemary oil is produced from rosemary and olive oil.

The hair and scalp treatment solution of the present invention does not require animal testing, does not include sulfates, gluten, paraffin, parabens, phthalates or DEA, and is readily absorbed into the scalp tissue. The hair and scalp treatment solution is a safe all-natural composition for hair growth and stimulation, wherein the solution is derived from naturally occurring sources, is unrefined and unprocessed. The hair and scalp treatment solution helps to regenerate damaged or dormant hair follicles, nourish the scalp and hair, and promote new hair growth.

The hair and scalp treatment solution may be marketed and sold in containers of various sizes and weights (e.g., 6 ounces or 8 ounces) in most natural food health stores, where consumer care products are typically sold over the internet, catalogs or other retail outlets. Once acquired, the individual can apply the hair and scalp treatment solution thoroughly onto the scalp, and allow the same to remain on the scalp for a minimum of twenty or thirty minutes, or as long as overnight. The treatment may then be washed or rinsed out as part a normal hair care routine.

To the accomplishment of the foregoing and related ends, certain illustrative aspects of the disclosed innovation are described herein in connection with the following description and the annexed drawings. These aspects are indicative, however, of but a few of the various ways in which the principles disclosed herein can be employed and are intended to include all such aspects and their equivalents. Other advantages and novel features will become apparent from the following detailed description when considered in conjunction with the drawings.

### BRIEF DESCRIPTION OF THE DRAWINGS

The description refers to provided drawings in which similar reference characters refer to similar parts throughout the different views, and in which:

FIG. 1 illustrates a diagrammatic representation of one potential embodiment of the ingredients of the hair and scalp treatment solution of the present invention in accordance with the disclosed architecture;

FIG. 2 illustrates a perspective view of one potential embodiment of the hair and scalp treatment solution of the present invention being removed from its container and applied to the scalp of a user in accordance with the disclosed architecture;

FIG. 3 illustrates a perspective view of one potential embodiment of the hair and scalp treatment solution of the present invention in various containers and ready for use by a user in accordance with the disclosed architecture;

FIG. 4 illustrates before and after perspective views of a portion of a scalp to which applications of the hair and scalp treatment solution of the present invention have been made in accordance with the disclosed architecture; and

FIG. 5 illustrates a perspective view of one potential embodiment of a logo or advertisement that can be used to market the hair and scalp treatment solution of the present invention.

### DETAILED DESCRIPTION OF THE INVENTION

The innovation is now described with reference to the drawings, wherein like reference numerals are used to refer to like elements throughout. In the following description, for purposes of explanation, numerous specific details are set forth in order to provide a thorough understanding thereof. It may be evident, however, that the innovation can be practiced without these specific details. In other instances, well-known structures and devices are shown in block diagram form in order to facilitate a description thereof. Various embodiments are discussed hereinafter. It should be noted that the figures are described only to facilitate the description of the embodiments. They are not intended as an exhaustive description of the invention and do not limit the scope of the invention. Additionally, an illustrated embodiment need not have all the aspects or advantages shown. Thus, in other embodiments, any of the features described herein from different embodiments may be combined.

As noted above, there is a long felt need in the art for a hair and scalp treatment solution that treats hair loss caused by diseases, such as alopecia or other medical conditions, and that can be easily applied to the hair and scalp area of the individual to effectively enhance the quality of the individual's hair, as well as the condition of the scalp. Moreover, there is a long felt need in the art for a hair and scalp treatment solution that prevents hair from becoming damaged due to over processing of the same, and that stimulates hair growth. Additionally, there is a long felt need in the art for a hair and scalp treatment solution that is all natural, and free of harsh chemicals and parabens. Finally, there is a long felt need in the art for a hair and scalp treatment solution that is inexpensive to manufacture, and that is cost-effective and affordable to treat hair loss, damaged hair and related conditions.

The present invention, in one exemplary embodiment, is a novel hair and scalp treatment solution comprised of an aloe vera gel, turmeric root, ginger root, petroleum jelly, grape seed oil, coconut oil, peppermint oil, rosemary oil, grave sea oil and a vanilla fragrance oil. The solution may further comprised a tint or natural color added thereto such as to form an amber, tan or cream colored mixture that stimulates hair growth and reduces alopecia. The hair and scalp treatment solution of the present invention preferably has a specific gravity ranging between 1.2 and 1.6 g/ml, and a viscosity ranging from between 4,000 and 7,000 cps.

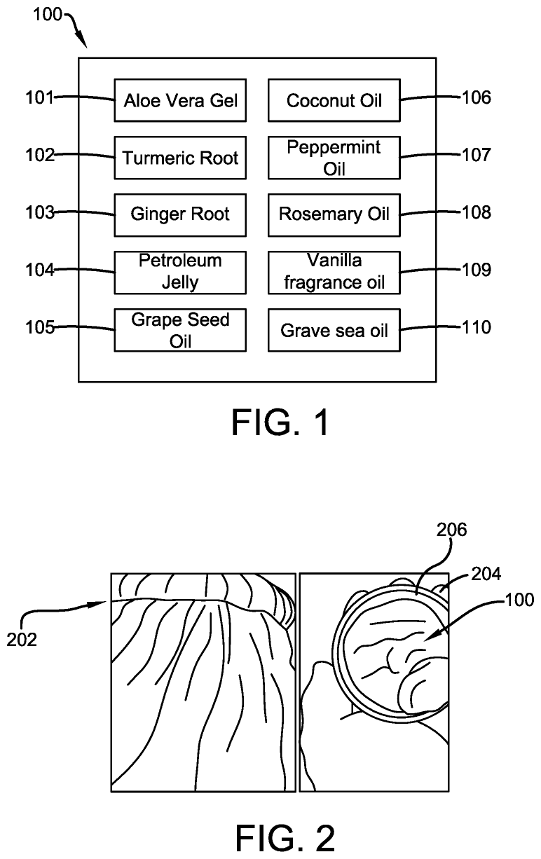

Referring initially to the drawings, FIG. 1 illustrates a diagrammatic representation of one potential embodiment of the ingredients of the hair and scalp treatment solution 100 of the present invention in accordance with the disclosed architecture. More specifically, the hair and scalp treatment solution 100 of the present invention is comprised of all natural ingredients which are derived from naturally occurring sources. All the ingredients give a unique quality and increased effectiveness of the hair and scalp treatment solution 100, thereby stimulating hair growth, reducing alopecia, and strengthening and conditioning hair.

More specifically, the aloe vera gel 101 conditions and improves damaged and dry hair, promotes hair growth, prevents itching of the scalp, reduces dandruff and overall conditions the scalp and hair of the individual. The turmeric root 102 revitalizes the scalp and also conditions the individual's hair and scalp, and the addition of ginger root 103 stimulates the individual's scalp for increased blood flow, which in turn helps promote hair regrowth. The inclusion of petroleum jelly 104 helps to reduce and/or prevent dryness of the individual's scalp, and provides a relative greasy texture to the solution 100, which prevents trans epidermal water loss.

The inclusion of the grape seed oil 105 acts as a natural dandruff treatment, and the coconut oil 106 has vitamins and essential fatty acids that nourish the scalp and helps to remove sebum build-up from the individual's hair follicles. The coconut oil 106 also helps to prevent protein loss from the hair and the scalp, and the peppermint oil 107 increases the number of follicles, the follicle depth, and overall hair growth, and also acts as a vasodilator to improve blood flow to the scalp. The inclusion of the rosemary oil 108 promotes nerve growth, improves circulation, and prevents dry or itchy scalp, while the vanilla fragrance oil 109 has antibacterial properties to help in treatment of the scalp. Grave sea oil 110 is also preferably included.

The essential oils used as ingredients in the hair and scalp treatment solution 100 are preferably extracted from plants, and are biologically able to promote hair growth. The essential oils are also excellent for cleansing, nourishing, and strengthening the hair follicles and shafts. Notwithstanding the forgoing, other essential oils that may help aid with the relaxation of the scalp and the individual include, but are not limited to lavender, rose, pumpkin, jojoba, eucalyptus, frankincense, cinnamon leaf, lime, cedarwood and lemongrass. These essential oils may be added in an amount of 0.5% to about 3% by weight depending on the essential oil and its potency. The formulation may also include vitamin supplements such as, but not limited to, omega 3 and 6, zinc, vitamin C, vitamin D, iron, B-5 and biotin, which may be added to increase the growing phase of the hair. Further, the carrier oils included in the formulation, such as coconut, rosemary, and peppermint, help to dilute the essential oils. Nonetheless, other carrier oils, such as sunflower, castor and mineral oil may also be used.

The hair and scalp treatment solution 100 of the present invention can strengthen hair, prevent hair loss, promote hair growth and provide relief to individuals from undesirable conditions, such as severe dryness, bumps, and scabs on the scalp. Table 1 discloses one potential embodiment of a hair and scalp treatment solution 100 comprised of a plurality of different ingredients in varying quantities in order to achieve the above stated objectives.

| Component | Percentage by weight (% w/w) |
|---|---|
| Aloe Vera gel | 3-5 |
| Turmeric root | 2-5 |
| Ginger root | 3-5 |
| Petroleum jelly | 10-20 |
| Grape seed oil | 2-5 |
| Coconut oil | 5-10 |
| Peppermint oil | 5-9 |
| Rosemary oil | 5-9 |
| Vanilla fragrance oil | 3-10 |
| Grave sea oil | 3-5 |
| Water | Remaining |

Table 2 discloses an alternative potential embodiment of a hair and scalp treatment solution 100 comprised of a plurality of different ingredients in varying quantities in order to achieve the above stated objectives.

| Component | Percentage by weight (% w/w) |
|---|---|
| Aloe Vera gel | 8-10 |
| Turmeric root | 4-5 |
| Ginger root | 2-4 |
| Petroleum jelly | 15-30 |
| Grape seed oil | 2-5 |
| Coconut oil | 10-15 |
| Peppermint oil | 5-9 |
| Rosemary oil | 5-9 |
| Vanilla fragrance oil | 3-10 |
| Grave sea oil | 3-5 |
| Water | Remaining |

Notwithstanding, the various ingredients of the solution 100 shown in each of Tables 1 and 2 above may be adjusted to accommodate for different individual hair types, conditions or other factors. Further, the hair and scalp treatment solution 100 may be provided as a topical application on the scalp of an individual, or may be dispersed via an applicator directly to the hair and scalp. The applicator may further comprise a brush or comb element on one end to help in massaging the solution into the scalp.

FIG. 2 illustrates a perspective view of one potential embodiment of the hair and scalp treatment solution 100 of the present invention being removed from its container 206 and applied to the scalp 202 of a user in accordance with the disclosed architecture. More specifically, the hair and scalp treatment solution 100 may be removed from the container 206 with the fingers 204 and gently applied or massaged into the individual's scalp 202. Alternatively, a spatula, applicator, brush or the like may be used to apply the hair and scalp treatment solution 100 to the scalp 202 of the individual. In still other embodiments, it is contemplated that the hair and scalp treatment solution 100 could also be applied to facial areas to grow a beard, eyebrows or mustache. The hair and scalp treatment solution 100 may also be tinted to provide color to the solution 100, is non-sticky, and is easily absorbed into the scalp or other skin areas.

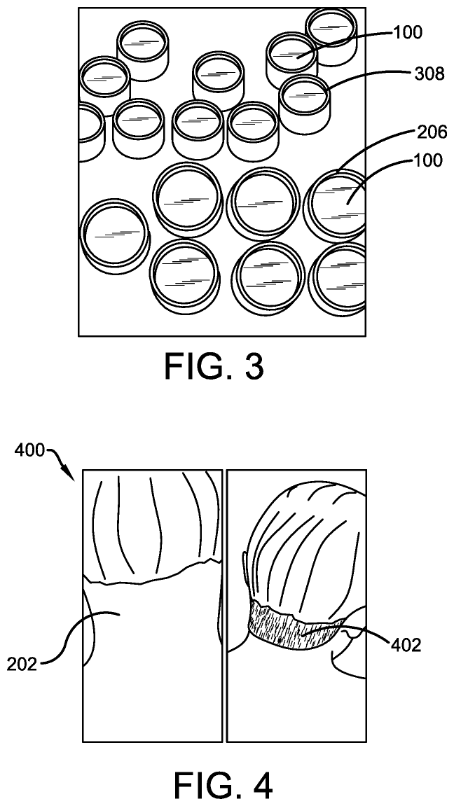

FIG. 3 illustrates a perspective view of one potential embodiment of the hair and scalp treatment solution of the present invention 100 in various sized containers and ready for use by a user in accordance with the disclosed architecture. More specifically, the hair and scalp treatment solution 100 may be made available in 4, 6, 8 and/or 10-ounce jars 308. Bigger jars 206 are also available as per the preferences of the consumers or retailers offering the product. Nonetheless, each of the jars 206, 308 are sealed with a lid to maintain the properties and texture of the hair and scalp treatment solution 100 until needed. The jars 206, 308 may further comprise logos, indicia, trademarks, geometric patterns, customizable colors and fonts, embroidery and prints and/or other images on their surface and lid. The jars 206, 308 may also be of any suitable shape including, but not limited to, rectangular, cubic, pyramidal, cylindrical, spherical, or any other shape. In addition, the jars 206, 308 may take on a promotional shape to coincide with a particular marketing or entertainment event. Accordingly, the jars 206, 308 shown in the various FIGS. and described the embodiments are not limited to any particular size or shape.

FIG. 4 illustrates before and after perspective views of portions of a scalp 202 to which applications of the hair and scalp treatment solution 100 of the present invention have been made in accordance with the disclosed architecture. More specifically, when a user 400 applies the hair and scalp treatment solution 100 of the present invention to the scalp 202 over a period of time, for example, two times a day consecutively for a minimum of three weeks or as otherwise recommended, the user should begin to see the regrowth of hair 402. The user applies a relatively small amount or thin layer of the hair and scalp treatment solution 100 to the scalp 202 and/or areas of the scalp 202 where treatment is needed. The application of the treatment 100 assists in reducing alopecia, treating over-processed hair and hair loss, as well as stimulating follicles to provide new hair growth.

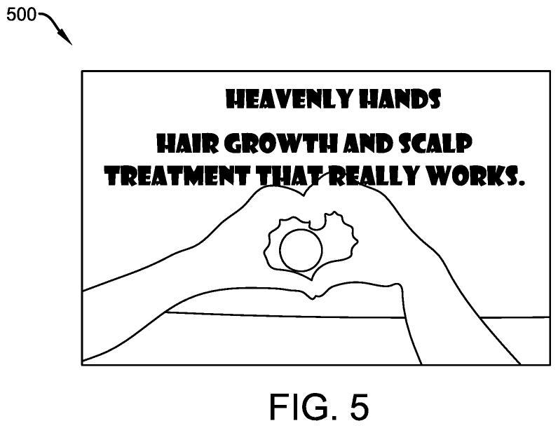

FIG. 5 illustrates a perspective view of one potential embodiment of a logo 500 or advertisement that can be used to market the hair and scalp treatment solution 100 of the present invention. For example, the hair and scalp treatment solution 100 may be marketed with a logo in amber color to signify the amber color of the solution 100. Also, one or more taglines, such as “HAIR GROWTH AND SCALP TREATMENT THAT REALLY WORKS” or any other tagline, to show the consumer the value of the hair and scalp treatment solution 100 may also be included subject to any regulatory requirements.

In yet another embodiment, a method for applying the hair and scalp treatment solution 100 is disclosed. The method includes the initial step of applying a relatively thin layer of the hair and scalp treatment solution 100 to the affected portion of the individual's scalp 202 or skin with the fingers 204 or a suitable applicator or brush. After a pre-determined time has passed, such as twenty to thirty minutes for a quick treatment or overnight for a longer treatment, the hair and scalp treatment solution 100 may be rinsed or washed off with water. Alternatively, the hair and scalp treatment solution 100 need not to be entirely rinsed off from the scalp 202, and a portion thereof may be allowed to be absorbed into the scalp 202.

The preferred embodiments of the present invention provide a hair care composition that is applied topically to the scalp or other areas of the skin. The treatment provides nourishment to the hair follicle, thereby promoting hair health and new hair growth. More particularly, the treatment helps to reverses hair loss by promoting new hair grow and increasing the rate of hair growth with regular use.

Certain terms are used throughout the following description and claims to refer to particular features or components. As one skilled in the art will appreciate, different persons may refer to the same feature or component by different names. This document does not intend to distinguish between components or features that differ in name but not structure or function. As used herein “hair treatment”, “hair grease”, “oil”, “cream”, and “treatment” are interchangeable and refer to the hair and scalp treatment solution 100 of the present invention.

Notwithstanding the forgoing, the hair and scalp treatment solution 100 of the present invention can contain any additional ingredient known in the art without affecting the overall concept of the invention, provided that it accomplishes the above-stated objectives. One of ordinary skill in the art will appreciate that the ingredients and composition of the hair and scalp treatment solution 100 as shown in the FIGS. are for illustrative purposes only, and that many other compositions are well within the scope of the present disclosure.

Various modifications and additions can be made to the exemplary embodiments discussed without departing from the scope of the present invention. While the embodiments described above refer to particular features, the scope of this invention also includes embodiments having different combinations of features and embodiments that do not include all of the described features. Accordingly, the scope of the present invention is intended to embrace all such alternatives, modifications, and variations as fall within the scope of the claims, together with all equivalents thereof.

What has been described above includes examples of the claimed subject matter. It is, of course, not possible to describe every conceivable combination of components or methodologies for purposes of describing the claimed subject matter, but one of ordinary skill in the art may recognize that many further combinations and permutations of the claimed subject matter are possible. Accordingly, the claimed subject matter is intended to embrace all such alterations, modifications and variations that fall within the spirit and scope of the appended claims. Furthermore, to the extent that the term “includes” is used in either the detailed description or the claims, such term is intended to be inclusive in a manner similar to the term “comprising” as “comprising” is interpreted when employed as a transitional word in a claim.

What is claimed is:

1. A hair and scalp treating solution comprising:
a gel;
at least root product;
a carrier oil; and
an essential oil.

2. The hair and scalp treating solution as recited in claim 1 and further comprising a petroleum jelly.

3. The hair and scalp treating solution as recited in claim 1, wherein the at least one root product is selected from a group consisting of a turmeric root and a ginger root.

4. The hair and scalp treating product as recited in claim 1, wherein the carrier oil is selected from a group consisting of a grape seed oil, a coconut oil, a peppermint oil, a rosemary oil, a castor oil, a mineral oil and a vanilla fragrance oil.

5. The hair and scalp treating solution as recited in claim 1, wherein the essential oil is selected from a group consisting of a lavender, a rose, a pumpkin, a jojoba, an eucalyptus, a frankincense, a cinnamon leaf, a lime, a cedarwood, a lemongrass, a turmeric, a ginger, a peppermint, a rosemary, a vanilla and a combination thereof

6. The hair and scalp treating solution as recited in claim 1, wherein the hair and scalp treating solution has a specific gravity ranging between 1.2 and 1.6 g/ml and a viscosity ranging from between 4,000 and 7,000 cps.

7. The hair and scalp treating solution as recited in claim 1 and further comprising a supplemental vitamin.

8. The hair and scalp treating solution as recited in claim 7, wherein the supplemental vitamin is one of a group consisting of an omega 3, an omega 6, a zinc, a vitamin C, a vitamin D, an iron, a vitamin B-5 and a biotin.

9. The hair and scalp treating solution as recited in claim 1, wherein the carrier oil comprises between 15-40% by weight of the hair and scalp treating solution.

10. The hair and scalp treating solution as recited in claim 1, wherein the gel is an aloe vera gel that comprises between 20-25% by weight of the hair and scalp treating solution.

11. The hair and scalp treating solution as recited in claim 2, wherein the petroleum jelly comprises between 10-20% by weight of the hair and scalp treating solution.

12. The hair and scalp treating solution as recited in claim 2, wherein the hair and scalp treating solution comprises about 3% by weight of the gel, about 7% by weight of the at least root product, about 10% by weight of the petroleum jelly, about 3% by weight of a grape seed oil, about 5% by weight of a coconut oil, about 5% by weight of a peppermint oil, about 5% by weight of a rosemary oil, about 7% by weight of a vanilla fragrance oil, and a quantity of water.

13. A hair and scalp treating solution comprising:
an aloe vera gel;
a turmeric root;
a ginger root;
a petroleum jelly;
a grape see oil;
a coconut oil;
a peppermint oil;
a rosemary oil;
a grave sea oil; and
a vanilla fragrance oil.

14. The hair and scalp treating solution as recited in claim 13, wherein the hair and scalp treating solution comprises between 3-5% by weight of the aloe vera gel, between 2-5% by weight of the turmeric root, between 3-5% by weight of the ginger root, between 10-20% by weight of the petroleum jelly, between 2-5% by weight of the grape seed oil, between 5-10% by weight of the coconut oil, between 5-9% by weight of the peppermint oil, between 5-9% by weight of the rosemary oil, between 3-10% by weight of the vanilla fragrance oil, between 3-5% by weight of the grave sea oil and a quantity of water.

15. The hair and scalp treating solution as recited in claim 13, wherein the hair and scalp treating solution has a specific gravity ranging between 1.2 and 1.6 g/ml and a viscosity ranging from between 4,000 and 7,000 cps.

16. The hair and scalp treating solution as recited in claim 13 and further comprising a supplemental vitamin.

17. The hair and scalp treating solution as recited in claim 16, wherein the supplemental vitamin is one of a group consisting of an omega 3, an omega 6, a zinc, a vitamin C, a vitamin D, an iron, a vitamin B-5 and a biotin.

18. The hair and scalp treating solution as recited in claim 13, wherein the hair and scalp treating solution comprises between 8-10% by weight of the aloe vera gel, between 4-5% by weight of the turmeric root, between 2-4% by weight of the ginger root, between 15-30% by weight of the petroleum jelly, between 2-5% by weight of the grape seed oil, between 10-15% by weight of the coconut oil, between 5-9% by weight of the peppermint oil, between 5-9% by weight of the rosemary oil, between 3-10% by weight of the vanilla fragrance oil and a quantity of water.

19. A hair and scalp treating solution comprising:
an aloe vera gel;
a turmeric root;
a ginger root;
a petroleum jelly;
a grape see oil;
a coconut oil;
a peppermint oil;
a rosemary oil;
a vanilla fragrance oil;
a grave sea oil; and
a supplemental vitamin, wherein the hair and scalp treating solution has a specific gravity ranging between 1.2 and 1.6 g/ml and a viscosity ranging from between 4,000 and 7,000 cps.

20. The hair and scalp treating solution as recited in claim 19, wherein the supplemental vitamin is one of a group consisting of an omega 3, an omega 6, a zinc, a vitamin C, a vitamin D, an iron, a vitamin B-5 and a biotin.
